(19) United States
(12) Patent Application Publication
(10) Pub. No.: US 2006/0069057 A1
(43) Pub. Date: Mar. 30, 2006

(54) Medicaments containing xenogeneic oligo- and/or polyribonucleotides

(76) Inventor: Hugo Seinfeld, Munchen (DE)

Correspondence Address: James R. Crawford;FULBRIGHT & JAWORSKI L.L.P., 666 Fifth Avenue, New York, NY, 10103, US

(21) Appl. No.: 11/250,067

(22) Filed: Oct. 13, 2005

Related U.S. Application Data
(63) Continuation of application No. 10/049,216, filed on Apr. 18, 2002.

(30) Foreign Application Priority Data
Aug. 27, 1999 (DE) 199 40 748.7

(51) Int. Cl. A61K 48/00

(52) U.S. Cl. 514/44

(57) ABSTRACT

The invention relates to medicaments that contain xenogenic oligo- or/and polyribonucleotides as the effective component. The invention further relates to the use of said xenogenic oligo- or/and polyribonucleotides for treating herpesviridae infections and skin tumors.

The invention relates to medicaments which contain xenogeneic oligo- and/or polyribonucleotides as active ingredient. It furthermore relates to the use of said xenogeneic oligo- and/or polyribonucleotides for the treatment of Herpesviridae infections and skin malignancies.

### BACKGROUND OF THE INVENTION

Viruses of the Herpesviridae family are pathogens which are common throughout the world and to which most vertebrates are susceptible. The most important human herpes viruses are herpes simplex virus 1 and 2 (HSV-1, HSV-2), varicella zoster virus (VZV) and human cytomegalovirus (HCMV). HSV causes, in immunocompetent individuals, lesions of the skin or mucosas, which can reappear as recurrences time and again with varying frequency. Various herpes viruses are distinguished according to the location of lesions, for example herpes labialis or herpes genitalis, etc.

Present methods of treatment for such viruses mainly aim at inhibition of viral replication, for example with Acyclovir, as [sic] known inhibitor of viral DNA polymerase. However, the virus can become resistant to Acyclovir with time and this is the case in particular for herpes simplex. In addition, although conventional agents can provide relief in the case of acute lesions, they cannot prevent recurrences effectively.

In the late 1960s and early 1970s, it was found in the context of transplant research, that tissue pretreated with xenogeneic heterogeneous nucleic acids or weak antigens had substantially increased antititers in various immunological examination methods. These results were confirmed further using a number of various antigens in in vitro and in vivo studies. However, there was no indication that nucleic acids and in particular oligo- and/or polyribonucleotides of xenogeneic origin could be suitable for controlling viral infections.

At the same time, especially in the USA, experiments with defined synthetic poly- and oligonucleotides, particularly ribonucleotides, were carried out, which, however, were not pursued any further, due to the high toxicity in vivo.

It was therefore the object of the present invention to provide a medicament which is suitable for the treatment of Herpesviridae infections and also of malignant skin disorders. It was furthermore an object of the invention to provide a medicament which reduces the recurrence rate for lesions of the skin, in particular for lesions caused by viruses.

According to the invention, the object is achieved by a medicament which comprises xenogeneic oligo- and/or polyribonucleotides as active substance.

Xenogeneic in accordance with the present invention means that the ribonucleic acid originates from an organism different from the one to be treated therewith, i.e. those oligo- and/or polyribonucleotides which are not from the same organism as that to which the medicament is to be administered. The xenogeneic oligo- and/or polyribonucleotides used according to the invention are preferably those from animal tissue (e.g. bovine tissue, fetal calf tissue), plants and unicellular organisms, preferably from yeast cells (in particular Saccharomyces cerevisiae). Preference is given to using oligo- and/or polyribonucleotides of organisms which are evolutionarily as distant as possible from the organism to be treated. Thus, in medicaments for humans preferably RNA from animal tissues or particularly preferably from plants or unicellular organisms such as, for example, yeast is used.

The invention is based on studies with RNA preparations in herpes infections. In this connection, it was found that applying isolated xenogeneic RNA to skin lesions of patients with herpes simplex labialis, herpes simplex cruris disseminata and herpes simplex genitalis, apart from the immediate action on the lesions themselves, in addition surprisingly also reduced significantly the recurrence rate in patients which had suffered over the year from frequently reappearing recurrences. It was then found that said RNA is active in a similar way also in the case of skin tumors, for example basaliomas.

The oligo- and/or polyribonucleotides used according to the invention are nontoxic and on their own nonantigenic.

It is possible to effectively use preparations of total RNA and salts and compounds thereof. Particular preference is given to tRNA. A particularly preferred manner of obtaining RNAs which can be used according to the invention is phenol extraction, specifically the methods denoted methods I and II herein.

The active amount of xenogeneic oligo- and/or polyribonucleotides per dosage depends in each patient on various factors, for example location of the lesions or size and extent of the affected area, and also type of administration. The dosage range is from 0.1 mg upward per dose unit. The lower limit of the amount per dose unit is preferably at least 0.5 mg, more preferably at least 2 mg, even more preferably at least 5 mg; and the upper limit is preferably 5 mg, more preferably 20 mg, even more preferably 10 mg.

The medicament of the invention preferably contains the xenogeneic oligo- and/or polyribonucleotides in essentially anhydrous form, for example as flakes, powder, granules, ointment or the like. However, the oligo- and/or polyribonucleotides may also be present in a soluble form in water or another solvent.

Additionally the medicament of the invention may comprise physiologically acceptable carriers, excipients, diluents and/or additives and/or adjuvants.

The pharmaceutical compositions which contain the xenogeneic oligo- and/or polyribonucleotides of the invention may be formulated for oral application as tablets, pastilles and chewable tablets, liquid suspensions, in powder form or as granules, emulsions, in hard or soft capsules, in syrup or elixir, as slow-release form or as osmotic capsules for slow release.

Another pharmaceutical form with particularly advantageous action is anhydrous ointments made of PEG mixtures.

Administration is carried out preferably topically, but also orally, parenterally, rectally or by inhalation. The term parenterally here relates to subcutaneous, intravenous, intramuscular and intrasternal injections or infusion techniques.

For topical application, the total RNA or tRNA used is preferably applied to the affected site as powder or PEG ointment (i.e. in anhydrous form); in the case of powder, the skin may be wetted slightly, where appropriate, and is preferably left to dry exposed to the air.

A particular embodiment of the invention is a medicament formulation for the treatment of disorders caused by Herpesviridae, which also reduces the frequency of recurrences in these disorders. The agent of the invention is particularly preferred for the treatment of lesions caused by herpes simplex viruses and herpes zoster (VZV), for example lesions and recurrences which are caused by herpes simplex labialis (herpes of the lips) and genitalis.

Xenogeneic oligo- and/or polyribonucleotides and the medicament of the invention are likewise suitable for treating skin malignancies such as, for example basaliomas.

The invention further relates to the use of said xenogeneic oligo- and/or polyribonucleotides for producing a medicament for the treatment of Herpesviridae disorders and skin tumors.

In the case of a lesion or a recurrence, a treatment is in each case preferably carried out as early as possible, and a single application already reduces the reappearance frequency.

In addition to treating humans with the xenogeneic oligo- and/or polyribonucleotides of the present invention, it is also possible to treat warm blooded animals such as, for example, horses, cattle, sheep, etc. in this way.

The following examples and experimental results further illustrate the invention.

### EXAMPLES

### Example 1

Production of the Oligo- and/or Polyribonucleotides Usable According to the Invention

The relevant literature describes a large number of methods for obtaining nucleic acids, nucleotides and nucleosides, which are known to anyone having the relevant experience. Two methods with small modifications, which are both based on phenolization, are preferably applied here, method I for obtaining the total RNA (Georgiev, G. P. and Mantieva, V. L., Biochim. Biophys. acta 61, 153 (1962)) and method II for obtaining the tRNA (Bauer, S. et al., Biotechnology and Bioengineering 15, 1081 (1973)). Both methods are suitable for extracting relatively large amounts.

Method I

A 15% suspension of brewer's yeast (Saccharomyces cerevisiae) was in buffer (A) [0.001 M EDTA, 0.01 M Tris-HCl buffer, pH 5-6, 25% sucrose, 0.5% SDS (sodium dodecyl sulfate), 0.3% Na deoxychlorate] was homogenized in a Waring Blendor [sic] at 10° C. and 3000 rpm for 3 minutes. The homogenate was admixed with the same volume of solution (B) [80% recrystallized phenol in buffer (A), 0.1% 8-hydroxyquinoline, 1.2% diethylpyrocarbonate] and then slowly stirred at 60° C. for 30 minutes. All buffer solutions were prepared with deionized water which had been agitated with bentonite beforehand.

The phenolized homogenate was then centrifuged at room temperature, approx. 20° C., and 10 000 g for 15 minutes. The aqueous phase was removed and the phenol and the intermediate phase were discarded. The aqueous phase was admixed with the same volume of a 1:1 mixture of solution (B) and chloroform/isoamyl alcohol (96:4) and extracted as described above. The aqueous phase was extracted three times with half the volume of diethyl ether in order to remove the remaining phenol. The solution was adjusted to 2% sodium acetate and the RNA was precipitated with 2.5 volumes of absolute ethanol.

The precipitated RNA was removed by centrifugation at 0° C. and 5 000 rpm and taken up in an ice-cold 0.01 M Tris-HCl buffer, pH 7.0 and 0.001 M MgCl₂. Possible DNA was degraded by adding electrophoretically pure pancreatic DNase (4 g/ml) to the solution and incubating at 22° C. for 3 hours. Protein residues, the DNase and RNases were digested with pronase (10 g/ml) at 37° C. for 3 hours. During this time, pronase was also destroyed by digesting itself. The RNA solution was extracted as described above with solution (B) at 60° C. with gentle stirring for 20 minutes, the phases were separated by centrifugation, the aqueous phase was removed and extracted with diethyl ether. After addition of sodium acetate (final concentration 2%), the RNA was precipitated with 2.5 volumes of ethanol and removed by centrifugation. The precipitate was taken up in cold 2% strength sodium acetate, precipitated with 2.5 volumes of ethyl alcohol and left in the alcohol mixture at −20° C. overnight. The precipitate was then removed by centrifugation, and washed twice with 75% strength ethanol, twice with absolute ethanol and twice with diethyl ether. After drying in an oven, a loose-packed dry RNA was obtained, which was stored in a dark glass vessel at room temperature.

Method II

This method is also suitable for extracting large quantities of yeast (kilogram quantities).

A given weight [sic] of yeast was homogenized in four times the amount of buffer (A) (see method I above) in the cold room. 40% v/v of phenol solution (B) and 5% w/v ice cubes made of deionized water were added to the homogenate and the mixture was stirred for 30 minutes. The supernatant was removed by suction and then phenolized two more times, as described under method I.

The aqueous supernatants were collected in a vessel which contained a DEAE-cellulose suspension (approx. 10% w/v, Whatman DE-22), corresponding to half the volume of the collected supernatants. The DEAE suspension was kept in suspension by stirring for 30 minutes. The DEAE was then allowed to sediment over one hour. The supernatant was removed by suction. In the meantime, the intermediate phase and phenol phase were stirred two more times with the aliquot amount of solution (C) (83% deionized water, 15% w/v ice cubes, 2% Mg-acetate concentrate [0.5M Mg-acetate in 0.25 [lacuna] mercaptoethanol] for 30 minutes and then allowed to separate for 70-80 minutes. The aqueous solutions were transferred into the vessel containing DEAE, and then again stirred and allowed to sediment. The supernatant was removed by suction and the DEAE was washed, as above, first twice with solution C, then again with solution (D) (2 volumes of Mg-acetate concentrate, 2 volumes of NaCl concentrate [3.75 M NaCl in water], 0.2 volumes of Tris-HCl concentrate [2.5 M Tris-HCl, pH 7.5 in water, 96 volumes of water]).

DEAE-cellulose was then packed into a column which was closed at the bottom. All further steps were carried out in the cold room at 4° C. The column was washed with 12 times the amount of the column contents of solution (D), flow rate 1.4 l/h, (only by gravity). The tRNA was then eluted with solution E [2 volumes of Mg-acetate concentrate, 0.2 volumes of Tris-HCl concentrate, 14 volumes of NaCl concentrate and 84 volumes of water, final NaCl concentration 0.525 M, with a flow of 3 l/h. The fractions which contained more than 35 A_{260 nm} units/ml were combined and precipitated with 1.5 volumes of ethanol. The further procedure was according to method I.

Alternatively, the final precipitate can be taken up in water and can be lyophilized.

A variant of this method is the common phenolization of the starting material: crude tRNA is precipitated out of the upper phase with isopropanol. After centrifugation, the precipitate is extracted with the sodium acetate buffer and chromatographed on DEAE-cellulose. Elution is carried out with a sodium acetate/sodium chloride gradient, as it is known to biochemists experienced in the subject-matter. The suitable fractions, see above, are determined by means of quotient measurement and combined. The tRNA is precipitated with ethanol, the precipitate is taken up as above and is preferably lyophilized.

The following assays were employed for analyzing the purity of the total RNA and tRNA and for characterizing them:

Protein was determined according to Lowry, O. H. et al. (J. Biol. Chem. 193, 265 (1951)) and by A₂₆₀/A₂₈₀≅2, DNA according to Dische (Mikrochemie 8, 4 (1930), total RNA according to Mejbaum (Physiol. Chem. 258, 117 (1939)), quantitative determination of tRNA and of amino acid incorporation according to Sprinzl and Sternbach (Methods in Enzymology 59, 182 (1979)) toxicity according to M. Nöldner (personal communication), absence of pyrogen in vitro according to DAB 1997 (LAL assay) and in vivo according to Ph. Eur./DAB 1997.

Results of the Analyses:

(Properties of Total RNA and tRNA, Averages from Ten Tests)

Absorption

A₂₆₀/A₂₈₀≅1.94-2.0

C, H, N analysis

| | | |
|---|---|---|
| C | 32.67 | 32.42 |
| H | 5.22 | 5.20 |
| N | 2.29 | 2.00 |

with corresponding values of various total RNAs and tRNAs.
UV and IR Spectra

The UV and IR spectra vary, they are almost the same but not identical, corresponding to biological substances.

Molecular Weight

Total RNA and tRNA from yeast ≅22 000-27 000 dalton average, varying for different preparations;

| Protein | DNA | (Total contents) |
|---|---|---|
| 2.3% | neg. | Total RNA of Saccharomyces cerevisiae |
| 1.9% | neg. | tRNA of Saccharomyces cerevisiae |
| 0.9% | neg. | Total RNA of bovine origin |

Average, generally common quality. Improved purity led to no significantly improved therapeutic action, at a disproportionally higher cost.

Amino Acid Incorporation for tRNA, Average of 10 Analyses

| | | |
|---|---|---|
| Lysine | 69-85 | pmol/A₂₆₀ unit |
| Phe | 41-55 | |
| Ser | 39-50 | |
| Val | 77-90 | |

These averages vary in yeasts of different lots within the range stated.

Toxicity

Test for acute toxicity in mice:
Animals: NMRI mice, male, Janvier, France
Administration: Intravenously into a tail vein
Observation period: 24 hours
Number of random samples: n=10 at highest concentration
Assay substance:
   a. bovine total RNA
   b. tRNA from brewer's yeast (Saccharomyces cerevisiae)
Solvent: 0.9% NaCl in water p.i.
   Result:

Up to a maximum dosage of 1 g/kg/10 ml i.v., the animals used in the test showed no conspicuous features whatsoever within the observation period of 24 hours.

Absence of Pyrogen

A. The pyrogen content of total RNA and tRNA, both as described previously, was determined using the in-vitro assay for endotoxins according to DAB 1997 (LAL TEST) and on rabbits according to Ph. Eur./DAB 1997.

1. Total RNA

Endotoxin standard EC 5
Amoebocyte lysate
   Sensitivity declared: 0.06 EU/ml
   Sensitivity found: 0.06 EU/ml

Test solution: 100 mg RNA dissolved in 20 ml of water-LAL (0.5%)

Result:

Endotoxin content of the test solution 0.5% 1:5 diluted with water-LAL: <0.03 EU/ml.

2. tRNA

Endotoxin standard EC 5
Amoebocyte lysate
   Sensitivity declared: 0.06 EU/ml
   Sensitivity found: 0.06 EU/ml

Test solution: 100 mg RNA dissolved in 20 ml of water-LAL (0.5%)

Result:

Endotoxin content of the test solution 0.5% 1:10 diluted with water-LAL: <0.03 EU/ml.

B. In vivo test for absence of pyrogen according to Ph. Eur./DAB 1997

1. Total RNA

Test solution 1% of assay substance in pyrogen-free water p.i.
Dose: 1.0 ml/animal
animals: 3 rabbits, corresponding to DAB 1997
   Result:

Sum of temperature differences of 3 rabbits was 1.05° C., thus pyrogens are not detectable.

2. tRNA

Test solution 1% of assay substance in pyrogen-free water p.i.
Dose: 1.0 ml/animal
animals: 2 times 6 rabbits, corresponding to DAB 1997
   Result:
a. Sum of temperature differences of 6 rabbits: 5.40° C.
b. Sum of temperature differences of 6 rabbits: 4.10° C., pyrogens detectable.

### Example 2

Detection of the Efficacy of the Substances of the Present Invention

70 Patients, 40 of these having herpes simplex I (H. labialis and 30 patients having herpes simplex II (H. genitalis), all having frequent recurrences, were treated with total RNA. The RNA came from extracts of bovine fetal tissue, with the exception of liver. The powder-like RNA was applied to the slightly wetted lesions, 5 to 10 mg, depending on the size of the lesion, and allowed to dry. All patients were observed for 1 year.

5 Patients were nonresponders with respect to recurrences, 7 patients could not be analyzed, due to insufficient compliance. All other patients who always had several recurrences per year showed a significant decrease in recurrences. The evaluation was carried out by means of the nonparametric Mann-Whitney U test. The significance of the results was p<0.001. (SPSS, Npar, Mann-Whitney U test).

In a double-blind study with an observation period of 1 year, two groups of in each case 100 patients having herpes simplex labialis and herpes simplex genitalis with more than 4 recurrences per year were treated with bovine total RNA as above or with tRNA from brewer's yeast. Evaluation was carried out after one year using the program SPSS, Npar TEST: Mann-Whitney and λ² test.

In comparison with the placebo patients, the reduction in recurrences was was [sic] highly significant: in both cases p<0.001. The difference between the two RNAs was not large.

These results justify the use of said RNA in patients, in particular since no side effects or toxic symptoms whatsoever could be observed over several years.

When applying the described substances to facial herpes simplex in patients which also had a facial basalioma, it was found that said basalioma receded. Therefore the indication of the medicine of the invention also includes malignancies.

1-14. (canceled)

15. A method for the treatment of Herpesviridae infections by and/or skin tumors in a subject comprising administering to the subject in need thereof an amount of at least 0.1 mg of xenogeneic oligo- and/or polyribonucleotides once per recurrence of symptoms.

16. A method for treating lesions of the skin and/or mucosa caused by Herpes simplex virus and/or Varicella zoster virus comprising administering a therapeutically effective amount of a pharmaceutical composition comprising xenogenic oligo- and/or polyribonucleotides to a subject in need thereof once per recurrence of symptoms.

17. A method for the treatment of infections by Herpesviridae and/or skin tumors, comprising administering an active amount of at least 0.1 mg of xenogeneic oligo- and/or polyribonucleotides in an anhydrous preparation once per recurrence of symptoms to a subject in need thereof.

18. A method for the treatment of infections by at least one of Herpesviridae or skin tumors comprising administering to a subject in need thereof a therapeutically effective amount of xenophobic oligo- and/or polyribonucleotides once per recurrence of symptoms.

19. The method of claim 18, wherein the therapeutically effective amount is at least 0.1 mg.

20. The method of claim 15, wherein said oligo- and/or polyribonucleotides are administered topically.

21. The method of claim 20, wherein said oligo- and/or polyribonucleotides are a powder.

22. The method of claim 15, wherein said oligo- and/or polyribonucleotides are administered topically.

23. The method of claim 22, wherein said oligo- and/or polyribonucleotides are a powder.

24. The method of claim 19, wherein said olin and/or polyribonucleotides are administered topically.

25. The method of claim 16, wherein said oligo- and/or polyribonucleotides are a powder.

26. The method of claim 15, wherein said xenogenic oligo- and/or polyribonucleotides are obtained from a plant, an animal or a unicellular organism.

27. The method of claim 16, wherein said xenogenic oligo- and/or polyribonucleotides are obtained from a plant, an animal or a unicellular organism.

28. The method of claim 17, wherein said xenogenic oligo- and/or polyribonucleotides are obtained from a plant, an animal or a unicellular organism.

29. The method of claim 26, wherein said oligo and/or polyribonucleotides are administered topically.

30. The method of claim 26, wherein said oligo- and/or polyribonucleotides are a powder.

31. The method of claim 27, wherein said oligo- and/or polyribonucleotides are administered topically.

32. The method of claim 31, wherein said oligo- and/or polyribonucleotides are a powder.

33. The method of claim 28, wherein said oligo- and/or polyribonucleotides are administered topically.

34. The method of claim 33, wherein said oligo- and/or polyribonucleotides are a powder.

35. The method of claim 15, wherein said xonogeneic oligo- and/or polyribonucleotides are obtained from Saccharomyces cerevisiae.

36. The method of claim 16, wherein said xonogeneic oligo- and/or polyribonucleotides are obtained from Saccharomyces cerevisiae.

37. The method of claim 17, wherein said xonogeneic oligo- and/or polyribonucleotides are obtained from Saccharomyces cerevisiae.

38. The method of claim 18, wherein said xonogeneic oligo- and/or polyribonucleotides are obtained from Saccharomyces cerevisiae.

39. A method for the treatment of a subject having a Herpesviridae infection and/or a skin tumor comprising administering to the subject an amount of xenogeneic RNA obtained by homogenizing cells containing RNA in a sucrose-containing buffer.

40. The method of claim 39, wherein the buffer is tris-HCl containing 0.14 MHCl and 0.25 M sucrose.

41. The method of claim 39, wherein the cells are Saccharomyces cerevisiae.

42. A method for treating lesions of the skin and/or mucosa caused by Herpes simplex virus and/or Varicella zoster virus comprising administering a therapeutically effective amount of a pharmaceutical composition comprising xenogenic RNA obtained by homogenizing cells containing RNA in a sucrose-containing buffer.

43. The method of claim 40, where the buffer is tris-HCl containing 0.14 MHCl and 0.25 M sucrose.

44. The method of claim 42, wherein the cells are Saccharomyces cerevisiae.

45. A method for the treatment of infections by Herpesviridae and/or skin tumors comprising administering a therapeutically effective amount of xenogeneic RNA obtained by homogenizing cells containing RNA in a sucrose-containing buffer.

46. The method of claim 45, wherein the buffer is tris-Hcl containing 0.14 MHCl and 0.25 M sucrose.

47. The method of claim 46, wherein the cells are Saccharomyces cerevisiae.

48. The method of claim 45, wherein the therapeutically effective amount is at least 0.1 mg.

49. The method of claim 42, wherein the therapeutically effective amount is at least 0.1 mg.

50. The method of claim 39, wherein said oligo- and/or polyribonucleotides are administered topically.

51. The method of claim 42, wherein said oligo- and/or polyribonucleotides are administered topically.

52. The method of claim 45, wherein said oligo- and/or polyribonucleotides are administered topically.

53. The method of claim 50, wherein said oligo- and/or polyribonucleotides are a powder.

54. The method of claim 51, wherein said oligo- and/or polyribonucleotides are a powder.

55. The method of claim 52, wherein said oligo- and/or polyribonucleotides are a powder.

56. The method of claim 39, wherein said xenogenic oligo- and/or polyribonucleotides are obtained from a plant, an animal or a unicellular organism.

57. The method of claim 42, wherein said xenogenic oligo- and/or polyribonucleotides are obtained from a plant, an animal or a unicellular organism.

58. The method of claim 45, wherein said xenogenic oligo- and/or polyribonucleotides are obtained from a plant, an animal or a unicellular organism.

59. The method of claim 39, wherein the RNA is tRNA.

60. The method of claim 42, wherein the RNA is tRNA.

61. The method of claim 45, wherein the RNA is tRNA.

62. A method for preventing a recurrence of a herpes virus infection comprising administering a single application of a therapeutically effective amount of anhydrous preparation of xenogeneic RNA prepared by extracting cells containing the RNA with phenol in the presence of sucrose to the affected area of a subject in need thereof.

63. The method of claim 15, wherein a Herpesviradae infection is treated.

64. The method of claim 15, wherein a skin tumor is treated.

65. The method of claim 16, wherein the lesion is caused by Herpes simplex.

66. The method of claim 16, wherein the lesion is caused by Varicella zoster.

67. The method of claim 17, wherein a Herpesviradae infection is treated.

68. The method of claim 17, wherein a skin tumor is treated.

69. The method of claim 39, wherein a Herpesviradae infection is treated.

70. The method of claim 38, wherein a skin tumor is treated.

71. The method of claim 42, wherein the lesion is caused by Herpes simplex.

72. The method of claim 42, wherein the lesion is caused by Varicella zoster.
